(19) United States
(12) Patent Application Publication
(10) Pub. No.: US 2007/0034011 A1
(43) Pub. Date: Feb. 15, 2007

(54) Method and apparatus for dynamic focusing in ultrasonic imaging

(76) Inventor: Pai-Chi Li, Taipei City (TW)

Correspondence Address: HDSL, 4331 STEVENS BATTLE LANE, FAIRFAX, VA, 22033, US

(21) Appl. No.: 11/187,875

(22) Filed: Jul. 25, 2005

(51) Int. Cl. A61B 8/00; G01N 29/04

(52) U.S. Cl. 73/606; 600/437

(57) ABSTRACT

A dynamic focusing apparatus and method for ultrasonic imaging, using transducer frequency encoding technique to map different positions of the surface of a transducer to different frequency responses. To a particular focal position, the time delay required for focusing from various positions on the transducer surface to the focal point can be calculated. By combining frequency responses and time delays that correspond to different transducer positions, a single transmit waveform can be used to drive the transducer and to realize focusing similar to that of an array imaging system. Similar principles can be applied to dynamic focusing at the receive end. A filter can be used to select particular positions on a transducer surface. The received signal extracted in this manner can also be time delayed for focusing. By combining all filters that correspond to different transducer positions, dynamic receive focusing can be implemented using a single position dependent filter.

### BACKGROUND OF THE INVENTION

1. Field of Invention

The present invention relates in general to an ultrasonic imaging device with dynamic focusing, and more particularly, to an ultrasonic imaging system applicable to biological and medical imaging and non-destructive testing.

2. Related Art

Ultrasonic imaging has been broadly applied in various fields, including biology, medicine and industry. Typically, the imaging system includes a transducer, a transmitter, a receiver, a signal processor and a display device. The image is displayed in a two-dimensional or three-dimensional format. To obtain good spatial resolution, the image can be formed by using an array of transducers, and focusing is achieved by controlling the waveform and the time delay of the signal for each channel. Focusing needs to be performed during both transmission and reception, and the precision is directly related to the image quality. Therefore, it is a very crucial part of imaging. In the two-dimensional image shown in FIG. 1, when the system is set up to focus at a point (x,y) located on the image plane, the difference between r′ (the distance from a particular channel to the focal point) and r (the distance from the center of the array to the focal point) is calculated as (i.e., r′−r). This difference can be divided by the speed of sound to obtain a time delay, such that the time required for transmit focusing (or receive focusing) for each channel can be determined. The same process is applied to all channels to eliminate the propagation path length difference. The other advantage of using an array is that the signal of each channel can be dynamically adjusted such that the system can focus to any point in the image plane. By extending such a concept with a two-dimensional array, any point in a three-dimensional space can be focused.

The above method requires a programmable imaging system such that optimal image quality can be achieved in various applications. However, this method requires a relatively complex system. For example, an imaging system with a 128 channel array and 64 system channels requires 64 sets of transmit and receive devices to independently control the waveform and the time delay for each channel. As the number of channels increases, both the cost and the complexity of the system increase.

Another method to form a two- or three-dimensional image requires mechanically scanning the transducer. In this case, the transducer is not partitioned into a plurality of independent channels. Therefore, the image is formed by using a fixed lens. The image quality out of the focal zone is significantly affected. In addition, the system performance is difficult to be programmed.

In U.S. Pat. Nos. 5,582,177 and 5,976,091, ultrasonic transducers with non-uniform thickness has been disclosed. These disclosures are for increasing the transducer bandwidth and determining the effective transducer size. However, dynamic focusing by properly designing of the transmit waveform and the receive filter is not disclosed at all.

### SUMMARY OF THE INVENTION

A dynamic focusing device for ultrasonic imaging, using a frequency encoded transducer with a pre-determined transmit waveform, dynamic focusing can be achieved using a filter at the receiver end.

In the dynamic focusing device, a frequency encoded technique is used to partition the transducer surface such that various areas of the transducer can be controlled using the frequency response. In other words, different positions on the encoded transducer can be independently and individually controlled by adjusting the frequency response of the transmitted waveform and the received signal. Thereby, coherent array imaging can be efficiently implemented.

When the transducer of the ultrasonic imaging system is fabricated from piezoelectric materials, the frequency encoding method can be implemented by varying thickness of the piezoelectric material. This is different from the conventional uniform thickness design for the transducer.

Dynamic focusing for ultrasonic imaging can thus be achieved using a simple system architecture. The device not only provides dynamic focusing for a single transducer imaging system, it can also be utilized to provide elevational focusing for an imaging system using one-dimensional arrays. In other words, the uniform slice thickness conventionally obtained from a two-dimensional array can now be obtained from a one-dimensional array.

The dynamic focusing device for ultrasonic imaging of the present invention uses the frequency encoding technique to map different positions on the surface of a transducer to different frequency responses. If the transducer is fabricated by piezoelectric materials, this can be achieved by changing the thickness of the piezoelectric materials. To a specific focal position, the time delay required for focusing to that position from the surface of the transducer can be calculated. By combining the various frequency responses with corresponding time delays, a single transmitter can be used to realize the focusing effect similar to that in array imaging using a one-dimensional array. The combined waveform is the signal that drives the transducer. The method can be similarly applied to dynamic focusing at the receiver end. The frequency response of a receive filter determines the positions on the transducer surface for the subsequent coherent sum. Focusing delays can also be applied to the received signal extracted in this manner for dynamic focusing.

### BRIEF DESCRIPTION OF THE DRAWINGS

The present invention can be further illustrated from the detailed description given below. The following illustrations do are not limit applications of the present invention:

FIG. 1 shows focusing for conventional two-dimensional imaging using time delays;

FIG. 2A shows a top view of an annular transducer;

FIG. 2B shows a side view of the transducer as shown in FIG. 2A;

FIG. 3 shows a top view of a rectangular transducer;

FIG. 4 shows a filter bank for dynamic receive focusing; and

FIG. 5 illustrates a single filter for dynamic receive focusing.

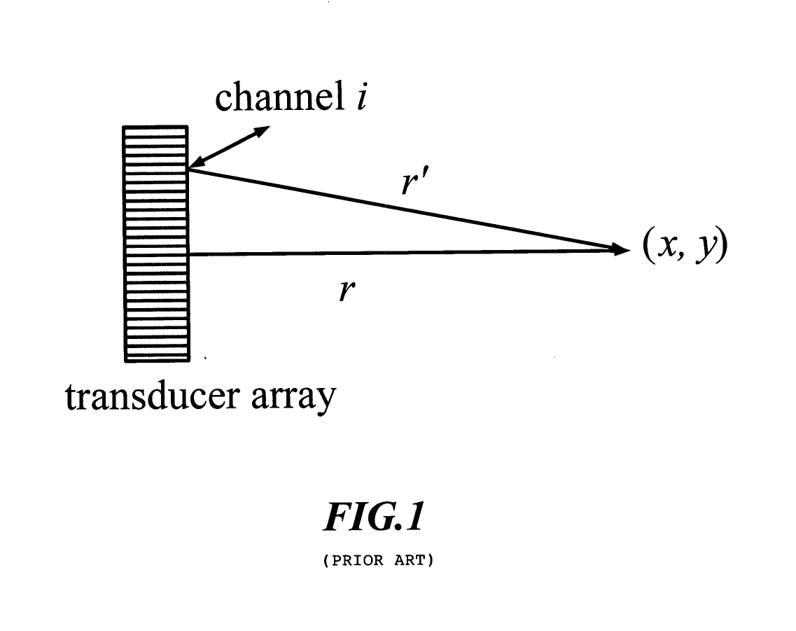

### DETAILED DESCRIPTION OF THE INVENTION

A dynamic focusing device for ultrasonic imaging includes an ultrasonic transducer, a transmitter and a receiver.

Typically, as the ultrasonic transducer is made of piezoelectric material, the frequency characteristic of the ultrasonic transducer is determined by the thickness of the piezoelectric material. The thickness of the piezoelectric transducer is about one half of the wavelength of the corresponding acoustic center frequency. As the attenuation of sound waves in a medium increases with the frequency, when the propagation depth is small, the signal frequency range can be higher than the frequency range with a larger propagation distance. Therefore, if the thickness is small at the central area and larger at the outside of the transducer, the effective aperture size is smaller at a nearer distance, and larger at a longer distance. Thereby, the image uniformity along non-scanning direction can be improved. Here, different thicknesses of transducer are designed corresponding to different frequency ranges. Therefore, signals at different frequency ranges can be used at the transmit end to control different positions on the transducer. Meanwhile, a filter can be used at the receive end to select the desired transducer area. Thus, dynamic focusing can be realized without using an array. That is, focusing from a one- or two-dimensional array can be achieved by using a single element transducer. That is, the system as provided requires only a single transmitter and a single receiver to achieve dynamic focusing. Therefore, the cost and complexity are greatly reduced.

Each transducer channel may correspond to one transmitter. Alternatively, multiple transducer channels may correspond to the same transmitter. The transmit waveform can be designed by using different frequency responses to map to different positions of the transducer. By delaying and summing the different frequency responses, focusing control can be performed at one or multiple positions simultaneously. The transmit signal can be designed as follows.

The transducer can be partitioned into N areas and each with a unique frequency response. Let the main operation frequency corresponding to the area i be denoted as ƒᵢ. Assuming the focal point is at (x,y) in the image plane, the required focusing time delay from a particular area to the focal point is denoted as τ(i,x,y). The template of the transmit waveform is denoted as p(t), and the following signals can be used to drive the transducer such that the transducer can automatically focus at the focal point:

$$s ( t ) = {\sum }_{i = 1}^{N} p ( t - τ ( i , x , y ) )$$

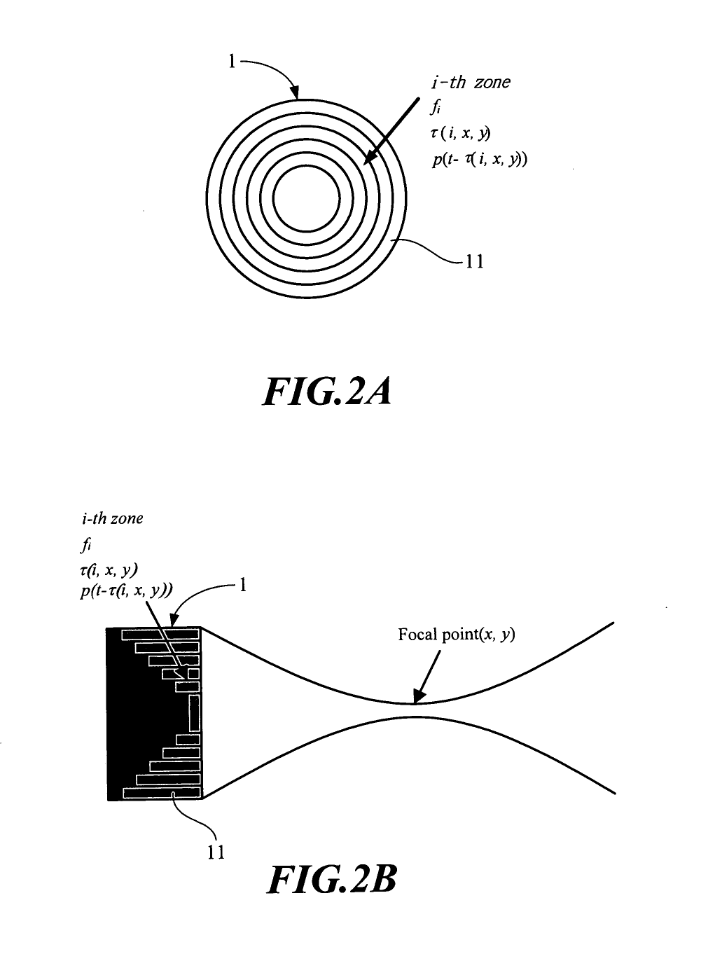

For example, for an annular transducer 1 shown in FIG. 2A, various rings 11 correspond to different frequency responses. As shown in FIG. 2B, the center area of the piezoelectric material of the annular transducer 1 is thinner, while the thickness of other rings 11 varies, thus producing different frequency responses. The thickness variations for different rings can be discrete or continuous.

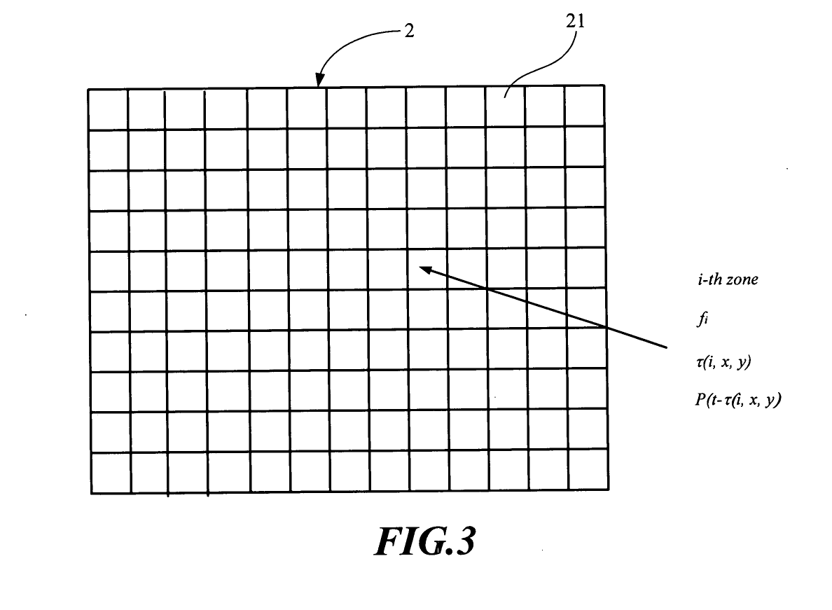

The present invention is not limited to the above transducer geometry, while other frequency encoding methods can also been implemented without exceeding the scope and spirit of the invention. For example, an equivalent two-dimensional array can be designed as shown in FIG. 3, in which each rectangular area 21 corresponds to a different frequency response.

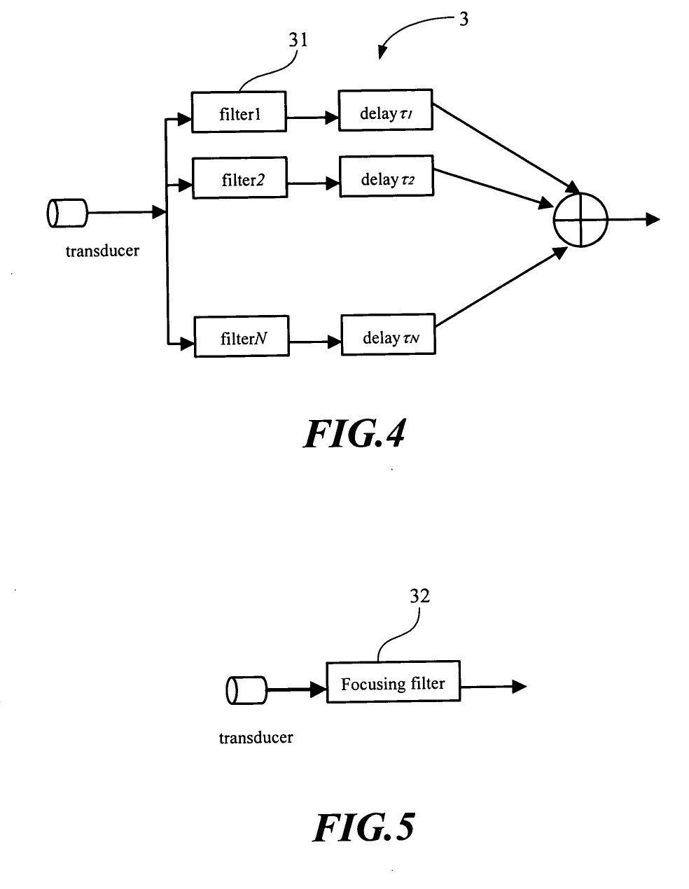

The working principle on receive is similar to that on transmit. A preferred embodiment of the receiver includes a set of band-pass filters with various frequency pass bands. In a linear system, this filter bank can be combined into a single filter. As shown in FIG. 4, in the receiver 3, each filter corresponds to a different transducer area. By imposing a time delay τ corresponding to each area prior to the summation, receive focusing can be implemented.

As the filter is linear and time invariant, the set of filters as shown in FIG. 4 can be combined into a single focusing filter 32, and the system structure is illustrated in FIG. 5. The operation can be represented as:

$$r ( t ) = {\sum }_{i = 1}^{N} e ( t ) * {h}_{i} ( t - {τ}_{i} ) = e ( t ) * h ( t ) ;$$

where * stands for the convolution operation, e(t) represents the received signal, hᵢ(t) and τᵢ are the frequency response and focusing delay required for the corresponding transducer area i.

Compared to conventional delay-and-sum methods, the dynamic focusing method as provided have following advantages.

First, the dynamic focusing method uses a frequency encoded transducer and a transmit waveform that can automatically produce one or multiple foci. Similarly at the receive end, a range dependent filter is used to achieve dynamic receive focusing.

Second, the frequency encoding method used by the dynamic focusing method divides the transducer into a plurality of areas and these areas can be controlled independently by tuning the frequency response of the transmit signal. In other words, by using the frequency encoded transducer, the frequency characteristics of the transmit waveform or the receive filter can be adjusted so as to independently control various transducer areas to obtain an image in a way similar to that performed by an array imaging system.

Third, frequency encoding of the transducer can be achieved by varying thickness of the piezoelectric material.

Moreover, the disclosed ultrasonic imaging method achieves dynamic focusing with reduced system complexity. In addition to improving the focusing quality of a single element transducer imaging system, the focusing method can also be extended to elevational focusing of a one-dimensional array imaging system. Using the disclosed method with a one-dimensional array, focusing quality of a conventional two-dimensional array imaging system can be achieved. Therefore, such a method can produce a uniform slice thickness throughout the image.

The invention being thus described, it will be obvious that the disclosed method and apparatus can be varied in many ways. Such variations are not to be regarded as a departure from the spirit and scope of the invention, and all such modifications as would be obvious to one skilled in the art are intended to be included within the scope of the following claims.

What is claimed is:

1. A dynamic focusing apparatus for ultrasonic imaging, including an ultrasonic transducer, a transmitter and a receiver, wherein the ultrasonic transducer has different frequency responses at different surface positions.

2. The apparatus of claim 1, wherein the ultrasonic transducer is a single transducer, a one-dimensional array or a two-dimensional array.

3. The apparatus of claim 1, wherein the transducer is fabricated from piezoelectric materials with non-uniform thickness across the transducer to correspond to different frequency responses thereof.

4. The apparatus of claim 3, wherein the thickness variations is continuous or discontinuous.

5. The apparatus of claim 3, wherein the ultrasonic transducer is annular and each ring has a different frequency response.

6. The apparatus of claim 1, wherein the ultrasonic transducer is connected to one transmitter or a plurality of transmitters.

7. The apparatus of claim 1, wherein the ultrasonic transducer is connected to one receiver or a plurality of receiver.

8. The method of claim 1, wherein the receiver includes a filter bank or a single filter.

9. The method of claim 8, wherein the frequency response of the filter corresponds a focal point or a group of focal points.

10. The method of claim 9, wherein the frequency response of the filter depends on the position of the focal point.

11. A dynamic focusing method associated with an dynamic focusing apparatus for ultrasonic imaging, wherein the dynamic focusing apparatus includes an ultrasonic transducer, a transmitter and a receiver, the method including the steps of transducer frequency encoding, transmit waveform design and receive filter design so that the ultrasonic transducer has different frequency responses at different surface positions.

12. The method of claim 11, wherein the transmit waveform is designed according to the specific frequency response of each partition of the ultrasonic transducer and incorporated with a corresponding focusing delay to perform focus control.

13. The method of claim 11, wherein one or a plurality of transducer partitions is driven by the same transmit waveform.

14. The method of claim 11, wherein the receiver includes a filter bank or a single filter.

15. The method of claim 14, wherein the frequency response of the filter corresponds a focal point or a group of focal points.

16. The method of claim 15, wherein the frequency response of the filter depends on the position of the focal point.

17. The method of claim 14, wherein the receiver is designed according to a harmonic frequency band that corresponds to nonlinear response of an image object.
